(19) United States
(12) Patent Application Publication
(10) Pub. No.: US 2023/0218238 A1
(43) Pub. Date: Jul. 13, 2023

(54) NONINVASIVE METHODS FOR QUANTIFYING AND MONITORING LIVER DISEASE SEVERITY

(71) Applicant: Mayo Foundation for Medical Education and Research, Rochester, MN (US)

(72) Inventors: Puru Rattan, Rochester, MN (US); Peter A. Noseworthy, Rochester, MN (US); Itzhak Zachi Attia, Rochester, MN (US); Paul A. Friedman, Rochester, MN (US); Douglas A. Simonetto, Rochester, MN (US); Vijay Shah, Rochester, MN (US); Joseph C. Ahn, Rochester, MN (US); Patrick S. Kamath, Rochester, MN (US)

(21) Appl. No.: 18/151,673

(22) Filed: Jan. 9, 2023

Related U.S. Application Data
(60) Provisional application No. 63/297,478, filed on Jan. 7, 2022.

(51) Int. Cl. A61B 5/00; G16H 10/60; G16H 50/30; G16H 50/20

(52) U.S. Cl. CPC A61B 5/7264; A61B 5/4244; G16H 10/60; G16H 50/30; G16H 50/20

(57) ABSTRACT

Systems and methods are provided for quantifying and monitoring liver disease in a patient from voltage-time data. A method comprises receiving voltage-time data of a subject, the voltage-time data comprising voltage data of a plurality of leads of an electrocardiograph; generating a feature vector from the voltage-time data; providing the feature vector to a pretrained learning system; receiving from the pretrained learning system a status of liver disease in the subject.

### RELATED APPLICATION(S)

This application claims the benefit of priority to U.S. Provisional Application No. 63/297,478, filed Jan. 7, 2022, which is incorporated herein by reference in its entirety.

### TECHNICAL FIELD

The invention relates generally to treatment of liver disease and, in particular, to systems and methods for quantifying and monitoring liver disease severity.

### BACKGROUND

Cirrhosis is a common endpoint in patients with chronic liver disease of various causes and is among the leading causes of morbidity and mortality globally. Many patients are first diagnosed when presenting advanced-stage complications. Over time, patients with cirrhosis develop progressive portal hypertension and loss of synthetic function, and transition from a relatively asymptomatic, “compensated” state to a “decompensated” state characterized by complications such as ascites, variceal hemorrhage, hepatic encephalopathy, hepatorenal syndrome, and infections. For patients with cirrhosis, accurate estimation of disease severity and prognosis is critical as it allows implementation of proper preventative and treatment measures as well as prioritization of organ allocation among candidates for liver transplantation (LT). One widely accepted model for end-stage liver disease (MELD) score includes laboratory markers of portal hypertension and hepatic dysfunction. However, the MELD score relies primarily on laboratory-based models with suboptimal performance and does not account for other physiological attributes.

Cirrhosis is associated with profound changes in the cardiovascular system including a hyperdynamic circulatory state and a form of cardiac dysfunction termed cirrhotic cardiomyopathy. Such physiologic changes are associated with distinct morphologic features on electrocardiograms (ECGs) of patients with cirrhosis, including prolonged QTc, low QRS voltage, decreased R-R interval variation, and short TpTe interval. These ECG changes are not mere observations, but closely related to the degree of hepatic decompensation and clinical outcomes in patients with cirrhosis. However, these ECG findings are generally subtle and non-specific, and have not been incorporated into routine evaluation and prognostication of patients with cirrhosis.

Accordingly, there is a need for a model for liver disease that incorporates additional variables representing different aspects of physiologic abnormalities that take plACEn patients with cirrhosis. As such, the Al-Cirrhosis-ECG (ACE) score system is disclosed herein. A deep learning-based artificial intelligence (AI) system utilizes a convolutional neural network (CNN) that processes digitized 12-lead ECGs and produces a numeric score between 0 and 1 whose magnitude reflects the estimated strength of cirrhosis-related ECG changes. In addition to having an excellent performance for determining the presence or absence of cirrhosis on ECGs, the ACE score is associated with markers of liver disease severity and mirrors the progression and resolution of liver disease in patients who undergo liver transplantation.

### SUMMARY

According to certain aspects of the present disclosure, systems and methods are disclosed for the diagnosis, scoring, and treatment of liver disease.

In one embodiment, a method comprises receiving voltage-time data of a subject, the voltage-time data comprising voltage data of a plurality of leads of an electrocardiograph; generating a feature vector from the voltage-time data; providing the feature vector to a pretrained learning system; receiving from the pretrained learning system a status of liver disease in the subject.

In another embodiment, a system comprises an electrocardiograph comprising a plurality of leads; a computing node comprising a computer readable storage medium having program instructions embodied therewith, the program instructions executable by a processor of the computing node to cause the processor to perform a method comprising: receiving voltage-time data of a subject, the voltage-time data comprising voltage data of a plurality of leads of an electrocardiograph; generating a feature vector from the voltage-time data; providing the feature vector to a pretrained learning system; receiving from the pretrained learning system a status of liver disease in the subject.

In another embodiment, a computer program product for detection of liver disease, the computer program product comprising a computer readable storage medium having program instructions embodied therewith, the program instructions executable by a processor to cause the processor to perform a method comprising: receiving voltage-time data of a subject, the voltage-time data comprising voltage data of a plurality of leads of an electrocardiograph; generating a feature vector from the voltage-time data; providing the feature vector to a pretrained learning system; receiving from the pretrained learning system a status of liver disease in the subject.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1 is a schematic view of a system for detecting liver disease, according to embodiments of the present disclosure.

FIG. 2 is a flowchart illustrating a method of detecting liver disease, according to embodiments of the present disclosure.

FIG. 3 is a diagram of an exemplary neural network model, according to embodiments of the present disclosure.

FIG. 4 is a flowchart illustrating cohort selection and model development, according to embodiments of the present disclosure.

FIG. 5 is a schematic of an exemplary computing node, according to embodiments of the present disclosure.

FIG. 6 is a graph depicting model performance, according to embodiments of the present disclosure.

FIG. 7 is a chart depicting subgroup analysis, according to embodiments of the present disclosure.

FIG. 8A is a chart depicting a relationship between MELD-Na score and ACE score.

FIG. 8B is a chart depicting longitudinal changes in ACE score before and after liver transplant.

FIG. 9 is a series of charts depicting the longitudinal changes in ACE score before and after liver transplant.

FIG. 10 is a chart depicting ACE scores among controls and early vs. advanced cirrhosis patients.

FIG. 11 is a schematic depicting an exemplary conceptual overview of the present disclosure.

FIG. 12 is a graph depicting an ROC curve for ACE score and hepatic decompensation.

FIG. 13 is a graph depicting ACE score distributions across ordinal outcomes in cirrhosis.

FIG. 14A is a graph depicting a Kaplan-Meier curve for probability of liver related mortality in the retrospective cohort of patients evaluated for liver transplantation.

FIG. 14B is a graph depicting a Kaplan-Meier curve for probability of liver transplantation in the retrospective cohort of patients evaluated for liver transplantation.

FIG. 15A is a graph depicting a Kaplan-Meier curve for probability of liver related mortality in the prospective cohort of patients evaluated for liver transplantation.

FIG. 15B is a graph depicting a Kaplan-Meier curve for probability of liver transplantation in the prospective cohort of patients evaluated for liver transplantation.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Cirrhosis of the liver may cause distinct cardiac dysfunction and electromechanical abnormalities that appear to correlate with the severity of liver disease. Convolutional neural networks (CNN) offer a comprehensive approach to analyzing and interpreting the vast amount of data generated in a single electrocardiogram (ECG), and more specifically, of the data generated by the electrical voltage of the ECG. Deep learning-based artificial intelligence (AI) models utilizing CNN have enabled automated prediction of subclinical left ventricular dysfunction, hypertrophic cardiomyopathy, atrial fibrillation, hyperkalemia as well as sex and age from digitized 12-lead ECGs. Structural and metabolic changes in the circulatory system that take place during cirrhosis may be reliably detected on a non-invasive, low-cost, and widely available 12-lead ECG by a CNN trained on a large and well-curated sample of cirrhosis patients. Aspects of the present disclosure include the development of a CNN using ECGs to distinguish patients with cirrhosis from controls without cirrhosis, producing a scoring system that correlates with the degree of disease severity, and predicting disease prognosis.

A review of Mayo Clinic's electronic health records identified 5,212 patients with advanced cirrhosis ≥18 years of age who underwent liver transplantation (LT) at three Mayo Clinic sites (MN; AZ; FL) between 1988 and 2019. The patients were matched on age and sex in a 1:4 ratio to controls without liver diseases, then divided into training, validation, and test sets using a 70%-10%-20% split. The primary outcome is the model's performance in distinguishing patients with cirrhosis from controls using ECGs. The secondary outcome is the association between the ACE score and the severity of patients' liver disease. The tertiary outcome is the model's ability to predict liver-related outcomes in patients with cirrhosis.

Algorithms can be developed using retrospective Mayo Clinic, patient-level data; including ECGs, procedural measurements, physician notes, and patient demographics for the purpose of screening patients for liver disease. These algorithms can be validated against MELD scores, manual assessments, and other suitable means discussed below.

All models may use voltage-time information from 12-lead ECGs as inputs. Modeling techniques may include convolutional neural networks with differing structures such as using all 12 leads as a single input, groups of 3 leads as separate inputs, each lead converted to spectrogram, and combinations of these methods. Additionally, the disease may also comprise underlying genetic components. The methods disclosed herein may be coupled with a genetic panel, to provide further specificity and sensitivity. Similarly, such methods may be used with genetic panels to detect novel biomarkers of disease.

Accordingly, in some aspects of the present disclosure, disclosed herein are methods comprising receiving voltage-time data of a subject, the voltage-time data comprising voltage data of a plurality of leads of an electrocardiograph; generating a feature vector from the voltage-time data; providing the feature vector to a pretrained learning system; and receiving from the pretrained learning system a status of liver disease in the subject. Generating the feature vector may comprise generating a spectrogram based on the voltage data of the plurality of leads. In some embodiments generating the feature vector comprises grouping the voltage data of the plurality of leads into a plurality of subsets.

In some embodiments, such methods further comprise receiving demographic information of the subject, wherein generating the feature vector comprises adding the demographic information to the feature vector. In some such embodiments, the learning system comprises a CNN. Such CNNs may comprise at least one residual connection.

In some embodiments the voltage-time data of a subject is received from an electrocardiograph. In further embodiments, the voltage-time data of a subject is received from an electronic medical record.

In some embodiments, the method further comprises providing the indication to an electronic health record system for storage in a health record associated with the subject. In some embodiments, the method further comprises providing the indication to a computing node for display to a user.

In some embodiments, the method further comprises generating a prediction of liver disease severity, outcome, or prognosis for the patient.

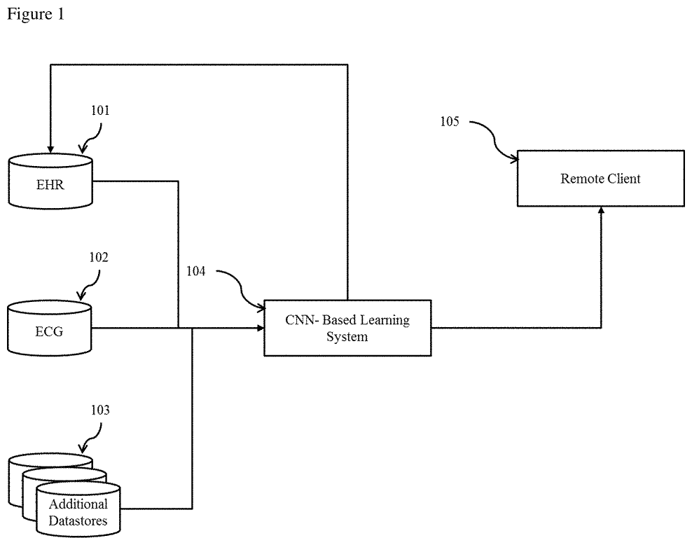

FIG. 1 is a schematic view of a system for detecting liver disease, according to embodiments of the present disclosure. As outlined above, in various embodiments, patient information, including electrocardiogram (ECG) data, is provided to a learning system in order to determine the presence of liver disease. Thus, aspects of the invention, as disclosed herein, also include a system comprising: an electrocardiograph comprising a plurality of leads; a computing node comprising a computer readable storage medium having program instructions embodied therewith, the program instructions executable by a processor of the computing node to cause the processor to perform a method comprising: receiving voltage-time data of a subject from the echocardiograph, the voltage-time data comprising voltage data of the plurality of leads; generating a feature vector from the voltage-time data; providing the feature vector to a pretrained learning system; and receiving from the pretrained learning system a status of liver disease (such as cirrhosis) in the subject. Generating the feature vector may comprise generating a spectrogram based on the voltage data of the plurality of leads. In some embodiments, generating the feature vector comprises grouping the voltage data of the plurality of leads into a plurality of subsets.

In some embodiments, such systems further comprise receiving demographic information of the subject, wherein generating the feature vector comprises adding the demographic information to the feature vector. In some such embodiments, the learning system comprises a convolutional neural network. Such convolutional neural networks may comprise at least one residual connection.

In some embodiments the voltage-time data of a subject is received from an electrocardiograph. In further embodiments, the voltage-time data of a subject is received from an electronic medical record.

In some embodiments, the system further comprises providing the indication to an electronic health record system for storage in a health record associated with the subject. In some embodiments, the system further comprises providing the indication to a computing node for display to a user.

Patient data may be received from electronic health record (EHR) 101. An electronic health record (EHR), or electronic medical record (EMR), may refer to the systematized collection of patient and population electronically stored health information in a digital format. These records can be shared across different health care settings. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information. EHR systems may be designed to store data and capture the state of a patient across time. In this way, the need to track down a patient's previous paper medical records is eliminated.

Electrocardiogram (ECG) data may be received directly from an electrocardiography device 102. In an exemplary 12-lead ECG, ten electrodes are placed on the patient's limbs and on the surface of the chest. The overall magnitude of the heart's electrical potential is then measured from twelve different angles (leads) and is recorded over a period of time (usually ten seconds). In this way, the overall magnitude and direction of the heart's electrical depolarization is captured at each moment throughout the cardiac cycle.

Additional datastores 103, may include further patient information as set out herein. Suitable datastores include databases, flat files, and other structures known in the art.

It will be appreciated that ECG data may be stored in an EHR for later retrieval. It will also be appreciated that ECG data may be cached, rather than delivered directly to a learning system for further processing.

Learning system 104 receives patient information from one or more of EHR 101, ECG 102, and additional datastores 103. As set out above, in some embodiments, the learning system comprises a convolutional neural network. In various embodiments, the input to the convolutional neural network comprises voltage-time information an ECG, which in some embodiments is paired with additional patient information such as demographics or genetic information.

Learning system 104 may be pretrained using suitable population data as set out in the examples in order to produce an indication of the presence or absence of liver disease (such as cirrhosis). In some embodiments, the indication is binary. In some embodiments, the indication is a probability value, indicating the likelihood of liver disease given the input patient data.

In some embodiments, learning system 104 provides the indication of liver disease for storage as part of an EHR. In this way, a computer-aided diagnosis is provided, which may be referred to by a clinician. In some embodiments, learning system 104 provides the indication of liver disease to a remote client 105. For example, a remote client may be a health app, a cloud service, or another consumer of diagnostic data. In some embodiments, the learning system 104 is integrated into an ECG machine for immediate feedback to a user during testing.

In some embodiments, a feature vector is provided to a learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

In machine learning, a convolutional neural network (CNN) is a class of feed-forward artificial neural networks applicable to analyzing visual imagery and other natural signals. A CNN consists of an input and an output layer, as well as multiple hidden layers. The hidden layers of a CNN typically consist of convolutional layers, pooling layers, fully connected layers and normalization layers. Convolutional layers apply a convolution operation to the input, passing the result to the next layer. The convolution emulates the response of an individual neuron to stimuli. Each convolutional neuron processes data only for its receptive field.

A convolution operation allows a reduction in free parameters as compared to a fully connected feed forward network. In particular, tiling a given kernel allows a fixed number of parameters to be learned irrespective of image size. This likewise reduces the memory footprint for a given network.

A convolutional layer's parameters consist of a set of learnable filters (or kernels), which have a small receptive field, but extend through the full depth of the input volume. During the forward pass, each filter is convolved across the width and height of the input volume, computing the dot product between the entries of the filter and the input and producing a 2-dimensional activation map of that filter. As a result, the network learns filters that activate when it detects some specific type of feature at some spatial position in the input.

In an exemplary convolution, a kernel comprises a plurality of weights w₁ . . . w₉. It will be appreciated that the sizes provided here are merely exemplary, and that any kernel dimension may be used as described herein. The kernel is applied to each tile of an input (e.g., an image). The result of each tile is an element of a feature map. It will be appreciated that a plurality of kernels may be applied to the same input in order to generate multiple feature maps.

Stacking the feature maps for all kernels forms a full output volume of the convolution layer. Every entry in the output volume can thus also be interpreted as an output of a neuron that looks at a small region in the input and shares parameters with neurons in the same feature map.

Convolutional neural networks may be implemented in various hardware, including hardware CNN accelerators and GPUs.

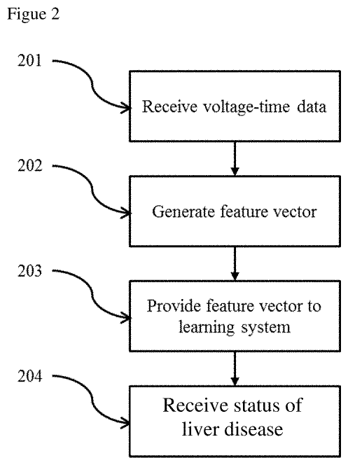

FIG. 2 is a flowchart illustrating a method of detecting liver disease, according to embodiments of the present disclosure. At 201, voltage-time data of a subject is received. The voltage-time data comprises voltage data of a plurality of leads of an ECG. The raw voltage-time data can be analyzed holistically, as opposed to using waveforms or features of the ECG itself as the input. This holistic analysis allows for patterns to emerge from the raw data that may not be noticeable in the ECG.

At 202, a feature vector is generated from the voltage-time data. The size of the feature vector is dependent on the input raw voltage-time data, where the feature vector matrix comprises rows corresponding to the input ECG leads and columns corresponding to time of the leads.

At 203, the feature vector is provided to a pretrained learning system. As discussed throughout the disclosure, the learning system can be a CNN or other suitable artificial neural network. The learning system may be trained on retrospective patient data and compared to ECG results throughout the patient's diagnosis and treatment.

At 204, an indication of the presence or absence of liver disease in the subject is received from the pretrained learning system. This indication can be a binary value, or a score generated by the pretrained learning system. The presence or absence of liver disease may be shown by a variety of hepatic decomposition events, including but not limited to ascites, spontaneous bacterial peritonitis, variceal hemorrhage, hepatic encephalopathy, and/or hepatorenal syndrome.

In some embodiments, association between the output indication score and hepatic decomposition at the time of the ECG can be assessed using univariable and multivariable logistic regression adjusting for MELD-Na and covariates including demographics, comorbidities, and etiology of liver disease.

For example, patients initially diagnosed with compensated cirrhosis can either develop hepatic decomposition or remain compensated. Step 204 of exemplary method 200 can indicate the presence of hepatic decomposition. The ability of the output of method 200 can be evaluated using receiver-operating characteristics (ROC) curve and the resulting area under the ROC curve (AUC). An optimal score cutoff for predicting decompensation can be determined using Youden's index to provide a balance between sensitivity and specificity.

In addition to the binary classification of a compensated vs. decompensated state, the distribution score can be evaluated across levels of ordinal outcomes in cirrhosis. An increase in score over time correlates to a worsening degree of hepatic decompensation.

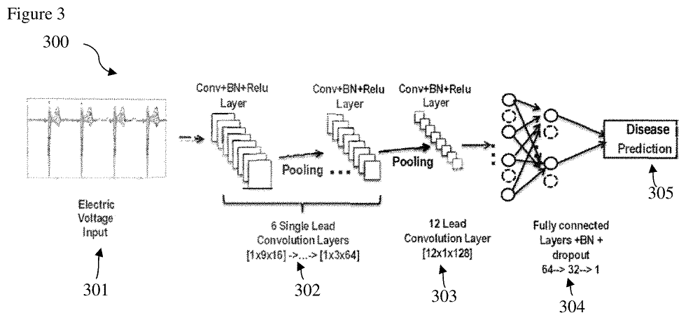

FIG. 3 is a diagram of an exemplary neural network model 300 for predicting a liver disease status from an electric voltage input 301. The model 300 processes an electric voltage input 301 from one or more channels of the patient's ECG. As shown, model 300 can include an input layer of perceptrons and/or sigmoid neurons, an output layer, and one or more hidden layers therebetween. For example, the data from multiple channels may be processed and pooled until the data is provided to a fully-connected layer of the network. The model 300 may output an estimated value or classification of a liver disease status for the patient.

In some implementations, the neural network model 300 includes multiple convolutional layers for feature extract. Each convolutional layer can include a constant or variable-length filter that focuses attention on the current lead or multiple leads of the ECG at the same time (e.g., k×1 or k×m leads). Following the convolutional layer, the network can include one or more fully connected layers with the same or different number of neurons in each. None, one, or more recurrent neural network layers can be added before, after, or in parallel to the convolutional layers for temporal feature extraction. Skip layers and neurons may or may not be included.

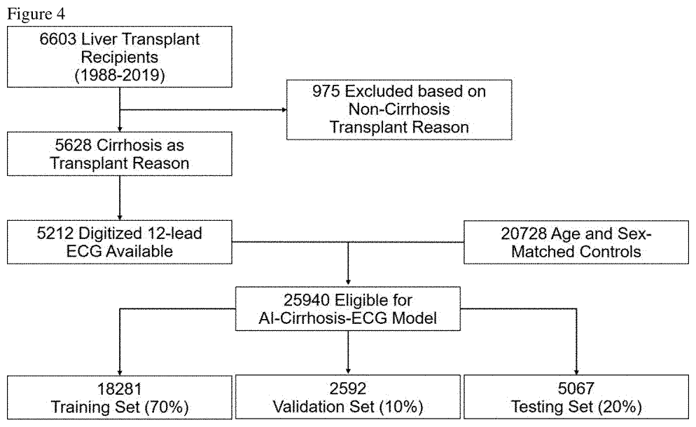

In an embodiment, a series of ECG inputs are converted to a matrix with 12 columns and 5,000 rows, each column representing one of the 12 leads from the ECG (representing the spatial dimension) and each row representing the lead amplitude in microvolts for that timestamp in each lead. For example, the ECGs may be sampled at 500 Hz. If the ECGs are of high quality, no pre-processing is required. The CNN architecture can have 9 convolutional blocks and two fully connected blocks, and can be trained from random initial values using an Adam optimizer. Multiple learning rates (ranging from 1×10⁻³ to 1×10⁻⁴ and multiple batch sizes and multiple batch sizes (16-64 samples) were used on the internal validation set and the rates with optimal area under the curve (AUC) (batch size of 32 and learning rate of 3×10⁻⁴ were used for testing the model on the holdout test set. To best capture cirrhosis-related ECG changes on the CNN, the model was trained using ECGs of patients with advanced cirrhosis who underwent liver transplantation. A retrospective review of Mayo Clinic's electronic health records was performed to identify patients over 18 years of age who underwent LT at three Mayo Clinic sites (MN; AZ; FL) between years 1988 and 2019 and had at least one standard, 10-second, 12-lead ECG at most a week prior to LT. Those who underwent LT for non-cirrhosis-related reasons were excluded. Patients were randomly matched on age and sex in a 1:4 ratio to controls without liver disease. Both the cirrhosis and the control cohorts were divided into training, validation, and test sets using a 70%-10%-20% split, as shown in FIG. 4. Baseline demographic information and medical comorbidities at the time of the ECGs were collected. Continuous variables were reported as mean (±SD) and median (Q1, Q3), and compared using the Student's t-test. Categorical variables were reported as absolute numbers and percentages and compared using the chi-square test.

In some embodiments, the model is trained and applied to two independent cohorts (retrospective and prospective) of patients with cirrhosis who received care at Mayo Clinic. The retrospective cohort included patients who were seen in the Division of Gastroenterology and Hepatology at Mayo Clinic, Rochester between Jan. 1, 1995, and Dec. 31, 2020, and first diagnosed with “compensated cirrhosis”. Patients with cardiac cirrhosis, hepatocellular carcinoma, or cholangiocarcinoma were excluded. Some of the patients in this retrospective cohort went on to develop decompensated cirrhosis resulting in death or liver transplantation, while others remained in the compensated state. The model was applied to all of the patient's ECGs from the time of first compensated cirrhosis diagnosis to the dates of their last follow up, liver-related death, or liver transplantation, producing a score for each ECG. For prospective validation of the score, a cohort of patients with advanced cirrhosis evaluated between Jan. 1, 2016 and Dec. 31, 2019 and prospectively recruited at the time of evaluation. All patients in this prospective cohort received an ECG at the time of evaluation, as well as laboratory studies necessary to calculate the MELD-Na score. Patients with non-cirrhotic etiologies of LT evaluation, cardiac cirrhosis, hepatocellular carcinoma, or cholangiocarcinoma were excluded. The model was applied to all of the patients' ECGs from the time of LT evaluation to the dates of their last follow up, liver-related death, or liver transplantation, producing a score for each ECG.

A binary classification model was trained using a CNN. The model input was a standard 10-second, 12-lead ECG and the output being the likelihood of the ECG being from a patient with cirrhosis or a prediction of liver disease progression. Each ECG was converted to a matrix with 12 columns and 5,000 rows, each column representing one of the 12 leads (spatial dimension) and each row representing the lead amplitude in microvolts for that timestamp in each lead (temporal axis, ECGs was sampled at 500 Hz). Both a Res-Net architecture (1, 2) and vanilla CNN (3, 4) were tested. As both had similar results on internal validation, a simple CNN was selected. The CNN's architecture includes 9 convolutional blocks and two fully connected blocks, and was trained from random initial values using the Adam optimizer. Multiple learning rates (ranging from 1×10⁻³ to 1×10⁻⁴) and multiple batch sizes (16-64 samples) were tested on the internal validation set and the ones that had the optimal area under the curve (AUC) (batch size of 32 and learning rate of 3×10⁻⁴) were used for testing the model on the holdout test set.

The primary outcome was the ability of the CNN to distinguish patients with cirrhosis from controls. The CNN produced the AI-Cirrhosis ECG (ACE) score, a continuous value between 0 and 1 indicating the estimated likelihood of cirrhosis on each ECG. After developing and refining the model, the receiver-operating characteristic (ROC) curve from the validation set was used to select an optimal ACE score threshold for binary classification. The CNN was then applied to a hold-out test set and its performance metrics were measured using this optimal threshold. To minimize confounding from other disease states, the CNN's performance in the test set was evaluated using controls matched not only for age and sex, but also for comorbidities, specifically hypertension, diabetes mellitus, cardiovascular disease, chronic kidney disease, and chronic lung disease. In addition, the model performance was assessed in subgroups of the test set categorized by sex, age, comorbid medical conditions, and etiologies of liver disease.

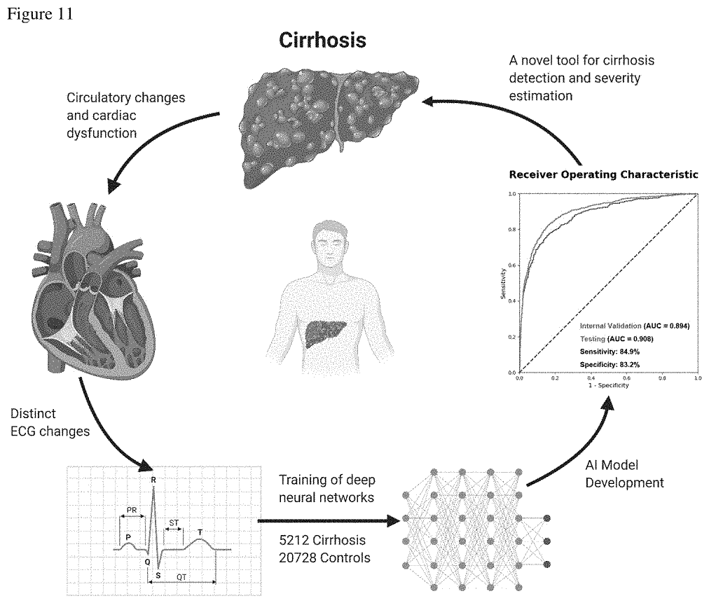

As disclosed herein, an AI model utilizing a CNN is able to differentiate between ECGs from patients with and without cirrhosis with excellent accuracy. The model's performance is maintained after matching for comorbidities including cardiovascular disease. The ACE score is also associated with liver disease severity as determined by the MELD-Na as well as individual laboratory markers. Furthermore, trends in the ACE score before and after LT reflect the progression and resolution of cirrhosis, and importantly does not appear related to post-transplant medication changes, including nonselective beta-blockers, diuretics, or lactulose. These results demonstrate that ECGs differ sufficiently between patients with and without cirrhosis to be discriminated by a CNN, and also that deep learning-based analyses of ECG signals offer promising potential as the basis of novel tools, such as the ACE score and its future iterations, in the care of patients with liver disease (FIG. 11).

Several mechanisms can play a role in the relationship between liver disease and ECG changes. Cirrhosis and the related development of portal hypertension are intricately linked to the circulatory system. Patients with cirrhosis develop a distinct type of cardiac dysfunction named cirrhotic cardiomyopathy independent of the etiology of liver disease. In patients with portal hypertension, there is increased production and activity of vasodilators such as nitric oxide, carbon monoxide, and endogenous cannabinoids within the splanchnic vasculature. This pathological state is known to have decreased vascular reactivity to vasoconstrictors. These agents may directly impact cardiac endothelium and myocytes, leading to subtle ECG changes. Additionally, these physiologic changes cause splanchnic vasodilation and a reduction in vascular resistance that leads to a compensatory hyperdynamic circulatory state characterized by an increased heart rate and cardiac output with low mean arterial pressure. These secondary compensatory changes may further impact the ECG.

The circulatory changes and cardiac dysfunction seen in patients with cirrhosis are not mere observations, but critically related to clinical outcomes. The hyperdynamic circulatory dysfunction and abnormal activation of vasoconstrictor systems such as the renin-angiotensin-aldosterone system, sympathetic nervous system, and antidiuretic hormone axis lead to hypervolemia and ascites. Persistent activation of vasoconstrictors can escalate into renal vasoconstriction resulting in hepatorenal syndrome, a deadly condition with high mortality. Studies suggest that cardiac dysfunction precedes hepatorenal syndrome and predicts poor survival in patients with cirrhosis. Furthermore, patients with cirrhosis-related cardiovascular dysfunction are at increased risk of decompensation following transjugular intrahepatic portosystemic shunt insertion as well as poor outcomes following LT.

Several electrophysiological changes in the context of cirrhosis have been studied. The most commonly reported ECG abnormality in cirrhosis is a prolonged QTc interval. In patients with cirrhosis, the prevalence of prolonged QTc interval increased with worsening Child-Pugh scores and significantly improved following LT. Proposed mechanisms for prolonged QTc in cirrhosis include increased sympathetic activity, molecular defects in the myocardial potassium and calcium channels, and increased levels of cardiotoxic substances due to portosystemic shunting. Other studies found significantly lower QRS voltage in patients with cirrhosis and noted its association with presence of ascites. R-R interval variations, considered to represent the integrity of the cardiac vagal nervous system, are also found to be decreased in patients with cirrhosis, especially in parallel with the presence and degree of hepatic encephalopathy. In addition, short TpTe intervals (the time from the peak to the end of the T wave) are found to be associated with disease severity and predictive of death and/or LT.

Despite the known electrophysiologic effects of cirrhosis, ECG findings are generally non-specific, often subtle, and highly variable. As such, they have not been incorporated into routine evaluation and management of patients with cirrhosis. This is the first instance to apply state-of-the-art deep learning-based AI methodologies to demonstrate the presence of a strong cirrhosis-associated ECG signal and quantify the signal in a manner that correlates with liver disease severity. The CNN is not constrained by the commonly known intervals and waves, but instead analyzes thousands of ECG waveforms to simultaneously process multiple, nonlinear, subtle patterns and combinations of features. The feasibility of this strategy is supported by demonstration of the ability of AI-ECG models to predict a variety of cardiac and non-cardiac conditions including left ventricular dysfunction, hypertrophic cardiomyopathy, paroxysmal atrial fibrillation, as well as hyperkalemia, sex, and age. Another strength of such a model is that ECGs are already used worldwide as one of the most ordered medical tests. Discovery of a new role for such an inexpensive, standardized, and ubiquitous test makes wide implementation feasible. Additionally, the fact that the model provided herein is able to achieve an excellent classification performance even with a single lead (lead I) is promising for its incorporation in app-based wearable devices.

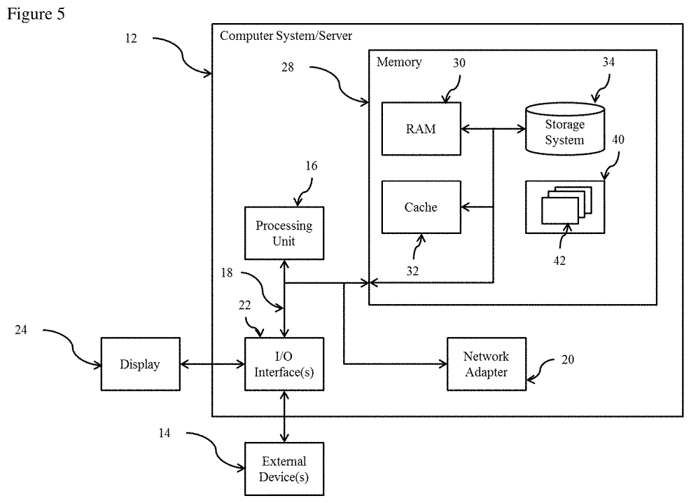

FIG. 5 is a schematic of an exemplary computing node. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. For example, in some aspects or the invention, provided herein is a computer program product for detection of cirrhosis, the computer program product comprising a computer readable storage medium having program instructions embodied therewith, the program instructions executable by a processor to cause the processor to perform a method comprising: receiving voltage-time data of a subject from the echocardiograph, the voltage-time data comprising voltage data of the plurality of leads; generating a feature vector from the voltage-time data; providing the feature vector to a pretrained learning system; and receiving from the pretrained learning system an indication of the presence or absence of liver disease in the subject. Generating the feature vector may comprise generating a spectrogram based on the voltage data of the plurality of leads. In some embodiments generating the feature vector comprises grouping the voltage data of the plurality of leads into a plurality of subsets.

In some embodiments, such computer program products further comprise receiving demographic information of the subject, wherein generating the feature vector comprises adding the demographic information to the feature vector. In some such embodiments, the learning system comprises a convolutional neural network. Such convolutional neural networks may comprise at least one residual connection.

In some embodiments the voltage-time data of a subject is received from an electrocardiograph. In further embodiments, the voltage-time data of a subject is received from an electronic medical record.

In some embodiments, the computer program product further comprises providing the indication to an electronic health record system for storage in a health record associated with the subject. In some embodiments, the system further comprises providing the indication to a computing node for display to a user.

The computer program product provided herein may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interfACEn each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### EXAMPLES

### Example 1: Relationship Between the ACE Score and Liver Disease Severity

The relationship between the magnitude of the ACE score and the severity of liver disease was evaluated as represented by the model for end-stage liver disease-sodium score (MELD-Na). For patients with cirrhosis in the test set, the patients' MELD-Na corresponding to the time of their ECGs was obtained. Individuals were then grouped into categories according to their MELD-Na, and the median ACE scores across these categories were compared using the Kruskal-Wallis test. In addition, the Spearman coefficients were calculated to assess correlation between the ACE score and laboratory markers of cirrhosis.

### Example 2: Longitudinal Changes in the ACE Score Before and After Liver Transplant

To assess how changes in the ACE score reflect the severity of patients' liver disease over time, longitudinal trends in the ACE scores of 547 test set patients who received long-term care at Mayo Clinic and had multiple 12-lead ECGs at various time points were investigated. The distribution of their ACE scores was assessed over time, categorized by years before and after liver transplantation (LT). The Kruskal-Wallis test was used to determine differences in median ACE scores between the time point categories.

### Example 3: ACE Scores in Asymptomatic Patients with Compensated Cirrhosis

As an additional validation, the performance of the model was tested in a distinct set of patients with earlier stages of disease, namely compensated cirrhosis. Review of the electronic records identified a cohort of 843 patients with cirrhosis without any decompensating events including variceal hemorrhage, ascites, or hepatic encephalopathy. ECGs were obtained for all these patients at most 6 months prior to the diagnosis of compensated cirrhosis according to clinical documentation. The distribution of ACE scores was compared to the original controls and patients with cirrhosis requiring LT.

### Example 4: Baseline Characteristics of the Patients with Cirrhosis and Controls

Table 1 demonstrates the baseline characteristics for the cirrhosis and control groups. Overall, there were 5,212 patients with cirrhosis meeting the inclusion criteria and 20,728 age and sex-matched controls without liver diseases. According to the 70%-10%-20% split, a total of 18,281 (3,665 cirrhosis vs. 14,616 controls), 2,592 (532 cirrhosis vs. 2060 controls), and 5,067 (1,015 cirrhosis vs. 4,052 controls) subjects were assigned to the training, validation, and test sets respectively (Table 2). All groups had comparable age (median age=57) and sex distributions (65% male). Controls had higher prevalence of cardiovascular diseases (coronary artery disease or cardiomyopathy) while the patients with cirrhosis had higher prevalence of diabetes mellitus, hypertension, chronic lung disease and chronic kidney disease. Among patients with cirrhosis, viral hepatitis was the most common cause of liver disease followed by alcohol-related liver disease, non-alcoholic steatohepatitis (NASH), biliary diseases, hereditary/genetic conditions, cryptogenic cirrhosis, autoimmune hepatitis, and other liver diseases. Hepatocellular carcinoma was present in 27.1% of patients.

TABLE 1
Baseline Characteristics

| | | Overall Set N = 25,940 | | | | |
|---|---|---|---|---|---|---|
| | | Cirrhosis | | Control | | p- |
| Categories | | N = 5,212 | | N = 20,728 | | value |
| Sex | F | 1,801 | (34.6%) | 7,207 | (34.7%) | 0.84 |
| | M | 3,411 | (65.4%) | 13,521 | (65.3%) | |
| Age | Median | 57.0 | (50.0, 63.0) | 57.0 | (50.0, 63.0) | 0.80 |
| | (Q1, Q3) | | | | | |
| | Mean | 55.63 | (10.17) | 55.66 | (10.17) | |
| | (SD) | | | | | |

| | | | | | |
|---|---|---|---|---|---|
| Cardiovascular | 458 | (8.8%) | 2,387 | (11.5%) | <0.01 |
| disease | | | | | |
| Diabetes mellitus | 1,793 | (34.4%) | 2,928 | (14.1%) | <0.01 |
| Hypertension | 2,268 | (43.5%) | 8,197 | (39.5%) | <0.01 |
| Chronic lung | 955 | (18.3%) | 3,452 | (16.7%) | <0.01 |
| disease | | | | | |
| Chronic kidney | 1,553 | (29.8%) | 1,453 | (7.0%) | <0.01 |
| disease | | | | | |
| Alcohol-related | 1,337 | (25.7%) | 0 | (0.0%) | <0.01 |
| liver disease | | | | | |
| NASH | 966 | (18.5%) | 0 | (0.0%) | <0.01 |
| Viral Hepatitis | 1,929 | (37.0%) | 0 | (0.0%) | <0.01 |
| (HBV/HCV) | | | | | |
| Biliary | 656 | (12.6%) | 0 | (0.0%) | <0.01 |
| (PBC/PSC) | | | | | |
| Autoimmune | 204 | (3.9%) | 0 | (0.0%) | <0.01 |
| hepatitis | | | | | |
| Hereditary/Genetic | 273 | (5.2%) | 0 | (0.0%) | <0.01 |
| (A1ATD, HH, WD) | | | | | |
| Cryptogenic | 238 | (4.6%) | 0 | (0.0%) | <0.01 |
| Hepatocellular | 1,413 | (27.1%) | 0 | (0.0%) | <0.01 |
| carcinoma | | | | | |
| Other liver | 302 | (5.8%) | 0 | (0.0%) | <0.01 |
| diseases | | | | | |

| | | | | |
|---|---|---|---|---|
| MELD | Median | 18.0 | (13.0, 24.0) | N/A |
| Score | (Q1, Q3) | | | |
| | Mean | 19.14 | (8.5) | N/A |
| | (SD) | | | |

Abbreviations:
A1ATD = alpha-1-antitrypsin deficiency;
HBV = hepatitis B virus;
HCV = hepatitis C virus;
HH = hereditary hemochromatosis;
PBC = primary biliary cholangitis;
PSC = primary sclerosing cholangitis;
WD = Wilson's disease

TABLE 2
Baseline Characteristics in the Training, Validation, and Test Sets

| | | Training Set | | | Validation Set | | | Test Set | | |
|---|---|---|---|---|---|---|---|---|---|---|
| | | N = 18,281 | | | N = 2,592 | | | N = 5,067 | | |
| | | Cirrhosis | Control | p- | Cirrhosis | Control | p- | Cirrhosis | Control | p- |
| Categories | | N = 3,665 | N = 14,616 | value | N = 532 | N = 2,060 | value | N = 1,015 | N = 4,052 | value |
| Sex | F | 1,273 | 5,115 | 0.77 | 178 | 695 | 0.90 | 350 | 1,397 | 0.96 |
| | | (34.7%) | (35.0%) | | (33.5%) | (33.7%) | | (34.5%) | (34.5%) | |
| | M | 2,392 | 9,501 | | 354 | 1365 | | 665 | 2,655 | |
| | | (65.3%) | (65.0%) | | (66.5%) | (66.3%) | | (65.5%) | (65.5%) | |
| Age | Median | 57.0 | 57.0 | 0.73 | 57.0 | 57.0 | 0.43 | 57.0 | 57.0 | 0.89 |
| | (Q1, Q3) | (50.0, 63.0) | (50.0, 63.0) | | (50.0, 62.0) | (50.0, 63.0) | | (50.0, 63.0) | (50.0, 63.0) | |
| | Mean | 55.77 | 55.71 | | 55.03 | 55.44 | | 55.47 | 55.52 | |
| | (SD) | (10.0) | (10.09) | | (10.92) | (10.48) | | (10.30) | (10.39) | |
| Cardiovascular | | 330 | 1,681 | <0.01 | 45 | 250 | <0.01 | 83 | 456 | <0.01 |
| disease | | (9.0%) | (11.5%) | | (8.5%) | (12.1%) | | (8.2%) | (11.3%) | |
| Diabetes | | 1,264 | 2,078 | <0.01 | 178 | 281 | <0.01 | 351 | 569 | <0.01 |
| mellitus | | (34.5%) | (14.2%) | | (33.5%) | (13.6%) | | (34.6%) | (14.0%) | |
| Hypertension | | 1,589 | 5,784 | <0.01 | 245 | 824 | 0.01 | 434 | 1589 | 0.04 |
| | | (43.4%) | (39.6%) | | (46.1%) | (40.0%) | | (42.8%) | (39.2%) | |
| Chronic lung | | 667 | 2,463 | 0.05 | 96 | 338 | 0.37 | 192 | 651 | 0.03 |
| disease | | (18.2%) | (16.9%) | | (18.0%) | (16.4%) | | (18.9%) | (16.1%) | |
| Chronic kidney | | 975 | 1032 | <0.01 | 149 | 135 | <0.01 | 281 | 286 | <0.01 |
| disease | | (26.6%) | (7.1%) | | (28.0%) | (6.6%) | | (27.7%) | (7.1%) | |
| Alcohol-related | | 941 | 0 | <0.01 | 139 | 0 | <0.01 | 257 | 0 | <0.01 |
| liver disease | | (25.7%) | (0.0%) | | (26.1%) | (0.0%) | | (25.3%) | (0.0%) | |
| NASH | | 682 | 0 | <0.01 | 99 | 0 | <0.01 | 185 | 0 | <0.01 |
| | | (18.6%) | (0.0%) | | (18.6%) | (0.0%) | | (18.2%) | (0.0%) | |
| Viral Hepatitis | | 1,345 | 0 | <0.01 | 178 | 0 | <0.01 | 406 | 0 | <0.01 |
| (HBV/HCV) | | (36.7%) | (0.0%) | | (33.5%) | (0.0%) | | (40.0%) | (0.0%) | |
| Biliary | | 461 | 0 | <0.01 | 75 | 0 | <0.01 | 120 | 0 | <0.01 |
| (PBC/PSC) | | (12.6%) | (0.0%) | | (14.1%) | (0.0%) | | (11.8%) | (0.0%) | |
| Autoimmune | | 129 | 0 | <0.01 | 28 | 0 | <0.01 | 47 | 0 | <0.01 |
| hepatitis | | (3.5%) | (0.0%) | | (5.3%) | (0.0%) | | (4.6%) | (0.0%) | |
| Hereditary/Genetic | | 196 | 0 | <0.01 | 24 | 0 | <0.01 | 53 | 0 | <0.01 |
| (A1ATD, HH, WD) | | (5.3%) | (0.0%) | | (4.5%) | (0.0%) | | (5.2%) | (0.0%) | |
| Cryptogenic | | 171 | 0 | <0.01 | 19 | 0 | <0.01 | 48 | 0 | <0.01 |
| | | (4.7%) | (0.0%) | | (3.6%) | (0.0%) | | (4.7%) | (0.0%) | |
| Hepatocellular | | 1,000 | 0 | <0.01 | 136 | 0 | <0.01 | 277 | 0 | <0.01 |
| carcinoma | | (27.3%) | (0.0%) | | (25.6%) | (0.0%) | | (27.3%) | (0.0%) | |
| Other liver | | 209 | 0 | <0.01 | 35 | 0 | <0.01 | 58 | 0 | <0.01 |
| diseases | | (5.7%) | (0.0%) | | (6.6%) | (0.0%) | | (5.7%) | (0.0%) | |
| MELD | Median | 18.0 | N/A | | 17.5 | N/A | | 18.0 | N/A | |
| Score | (Q1, Q3) | (13.0, 24.0) | | | (13.0, 24.0) | | | (12.0, 24.0) | | |
| | Mean | 19.21 | N/A | | 18.93 | N/A | | 19.14 | N/A | |
| | (SD) | (8.53) | | | (8.36) | | | (8.61) | | |

Abbreviations:
A1ATD = alpha-1-antitrypsin deficiency;
HBV = hepatitis B virus;
HCV = hepatitis C virus;
HH = hereditary hemochromatosis;
PBC = primary biliary cholangitis;
PSC = primary sclerosing cholangitis;
WD = Wilson's disease

### Example 5: Model Performance in the Test Set (ACE)

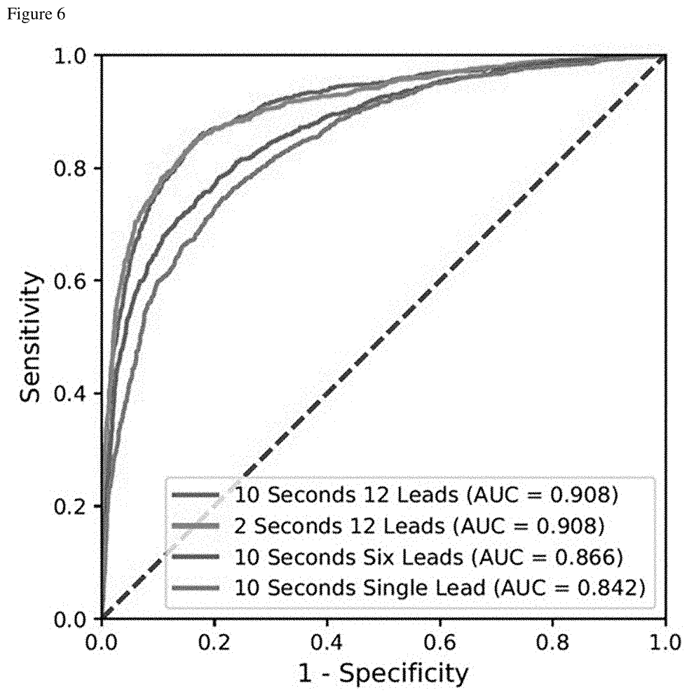

FIG. 6 shows the CNN's performance as a binary classifier within the test set. The receiver operating characteristic (ROC) curve for the model performance using 10 seconds of 12 leads, 2 seconds of 12 leads, 10 seconds of six limb leads (I, II, III, aVL, aVF, aVR), and 10 seconds of single lead (I). The full model using standard 10-second, 12-lead ECGs showed an excellent performance with AUC of 0.908. When the model was modified to use only the first 2 seconds of 12-lead ECGs, its AUC was unchanged at 0.908. Simpler models utilizing 10 seconds of six limb leads and 10 seconds of lead I continued to perform well with AUCs of 0.866 and 0.842, respectively. Using an ACE score of 0.17 as the optimal threshold, the model showed 84.9% sensitivity and 83.2% specificity, as well as 55.9% PPV and 95.7% NPV for the detection of cirrhosis in this cohort.

For the 1,015 patients with cirrhosis in the test set, 921 (90.7%) patients were matched in a 1:1 ratio to controls on 5 comorbid conditions of hypertension, diabetes mellitus, cardiovascular diseases, chronic kidney diseases, and chronic lung diseases (Table 3). In this matched subset of test set subjects, the ACE model's performance did not significantly change, with an AUC of 0.893, 83.6% sensitivity and 81.8% specificity.

TABLE 3
Distribution of comorbidities and ACE model performance in
the test set before and after matching for comorbidities

| | Before matching on comorbidities | | | After matching on comorbidities | | |
|---|---|---|---|---|---|---|
| | Cirrhosis | Controls | | Cirrhosis | Controls | |
| Comorbidities | (N = 1,015) | (N = 4,052) | p-value | (N = 921) | (N = 921) | p-value |
| Cardiovascular disease | 83 | 456 | <0.01 | 79 | 79 | 1.0 |
| | (8.2%) | (11.3%) | | (8.6%) | (8.6%) | |
| Hypertension | 351 | 569 | <0.01 | 408 | 408 | 1.0 |
| | (34.6%) | (14.0%) | | (44.3%) | (44.3%) | |
| Diabetes mellitus | 434 | 1589 | 0.04 | 278 | 278 | 1.0 |
| | (42.8%) | (39.2%) | | (30.2%) | (30.2%) | |
| Chronic kidney disease | 192 | 651 | 0.03 | 192 | 192 | 1.0 |
| | (18.9%) | (16.1%) | | (18.9%) | (18.9%) | |
| Chronic lung disease | 281 | 286 | <0.01 | 165 | 165 | 1.0 |
| | (27.7%) | (7.1%) | | (17.9%) | (17.9%) | |

| Model Performances | Before matching on comorbidities | After matching on comorbidities |
|---|---|---|
| AUC of the ACE model | 0.908 | 0.893 |
| Sensitivity of the ACE | 84.9% | 83.6% |
| model | | |
| Specificity of the ACE | 83.2% | 81.8% |
| model | | |

Abbreviations:
ACE = AI-Cirrhosis-ECG;
AUC = area under the curve

### Example 6: Subgroup Analyses

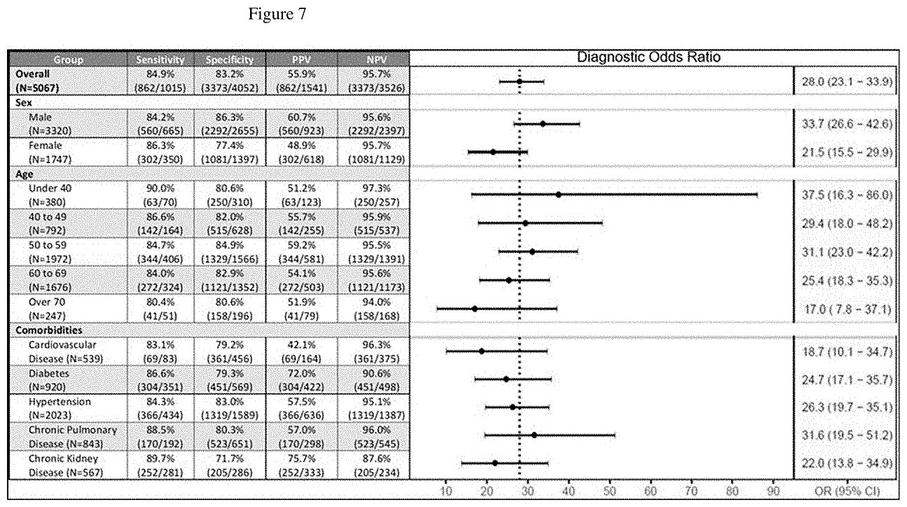

FIG. 7 shows the results of subgroup analyses on the test set according to age, sex, and medical comorbidities. The 95% confidence intervals for diagnostic odds ratios of all subgroups overlapped around the diagnostic odds ratio for the overall cohort, suggesting that the model performance was uniform regardless of subjects' sex, age, or comorbidities. Among patients with different etiologies of liver disease, the model's sensitivity was the highest in patients with NASH (92.4%) and consistently above 80% in patients with autoimmune, biliary, cryptogenic, viral, and other liver diseases. Notably, the sensitivity was lower for patients transplanted for hepatocellular carcinoma (73.6%).

### Example 7: Relationship Between ACE Score and Markers of Liver Disease Severity

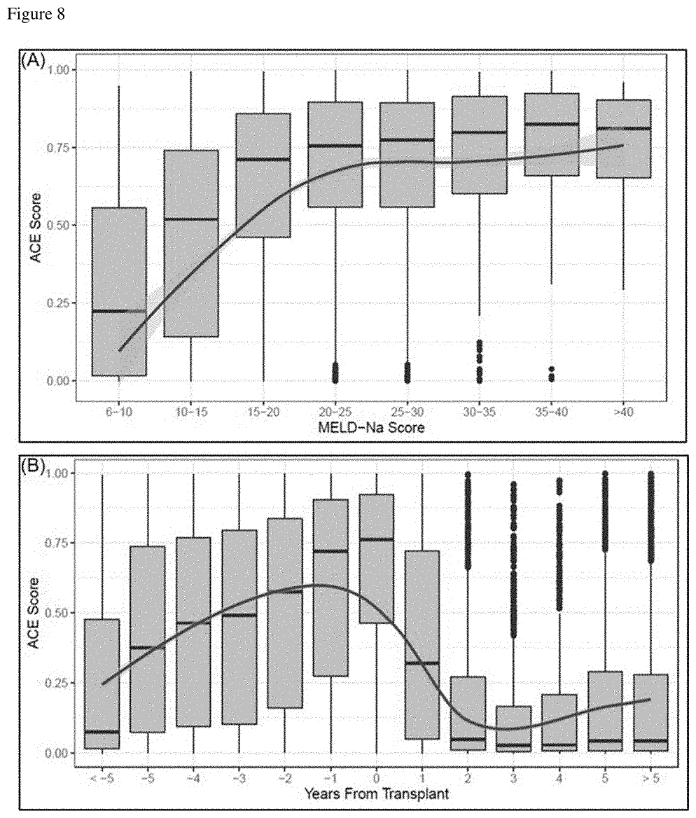

FIG. 8a shows the distributions of test set patients' ACE scores across groups of ascending MELD-Na. A clear trend of increasing ACE scores with increasing MELD-Na was observed for patients with MELD-Na less than or equal to 20. Starting at the lowest MELD-Na between 6 to 10, the median ACE scores were significantly higher compared to controls (0.225 vs. 0.016, p<0.001). Significant differences in ACE scores with rising MELD-Na groups of 6 to 10, 11 to 15, and 16 to 20 were found (median ACE: 0.225 vs. 0.519 vs. 0.713, p<0.001). A plateau in the ACE score was seen for MELD-Na above 20, with no significant differences in the high MELD-Na groups of 26 to 30, 31 to 35, 36 to 40, and above 40.

Assessing the Spearman's correlation coefficient (ρ) between the ACE score and several laboratory test-based markers of liver disease severity showed that the ACE score positively correlated with MELD-Na (ρ=0.3267, p<0.001), total bilirubin (ρ=0.2976, p<0.001) and INR (ρ=0.1120, p<0.001) (Table 4). On the other hand, an inverse correlation with platelet count (ρ=−0.2719, p<0.001) and serum sodium levels (ρ=−0.1757, p<0.001) was found.

TABLE 4
Correlation between ACE score and laboratory-
based markers of liver disease severity

| Variable | Spearman Correlation | P-Value |
|---|---|---|
| MELD-Na | 0.3267 | <0.001 |
| ALT | 0.0788 | 0.003 |
| AST | 0.0723 | 0.006 |
| Platelet count | −0.2719 | <0.001 |
| Creatinine | 0.0476 | 0.049 |
| Sodium | −0.1757 | <0.001 |
| Total bilirubin | 0.2976 | <0.001 |
| INR | 0.1120 | <0.001 |

Abbreviations:
ALT = alanine aminotransferase;
AST = aspartate aminotransferase;
FIB-4 = fibrosis-4 score;
INR = international normalized ratio;
MELD-Na = model for end-stage liver disease score-sodium

### Example 8: Changes in ACE Score Before and After Liver Transplant

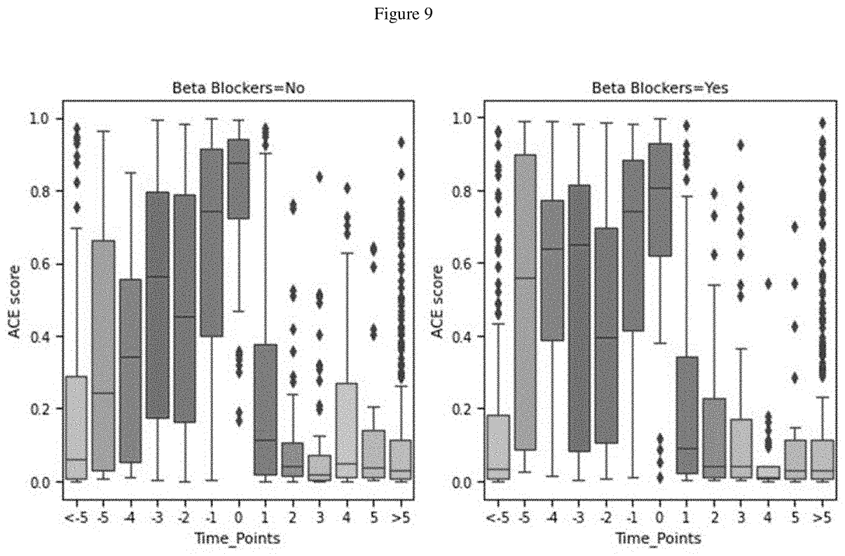

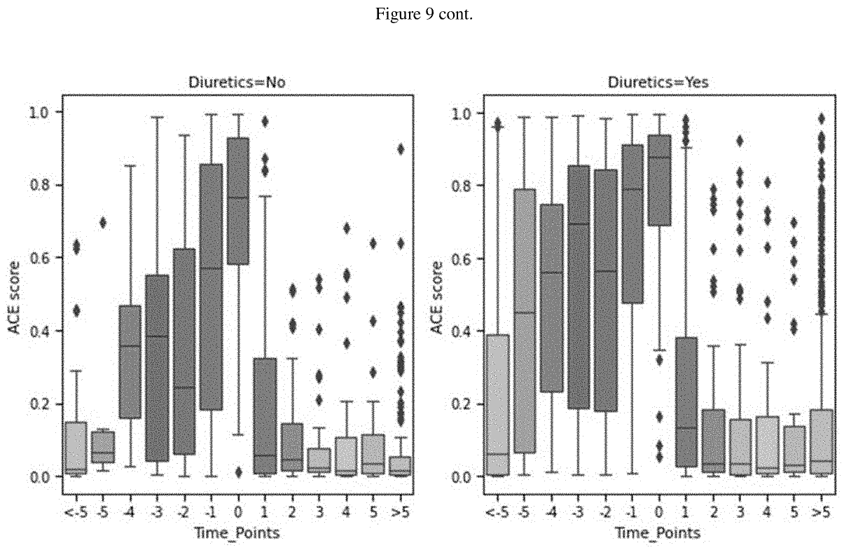

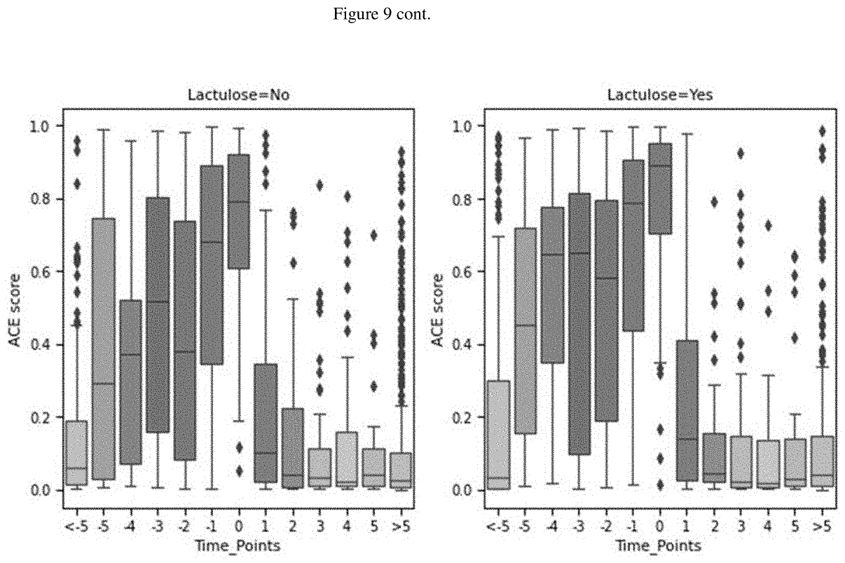

FIG. 8b shows the longitudinal changes in the ACE scores for 547 patients with cirrhosis who had ECGs at multiple time points prior to and after LT. Negative numbers represent years prior to transplant, positive numbers represent years following transplant. Significant increases in median ACE scores were observed year-over-year leading to the time of LT, starting around 0.079 more than five years pre-transplant and rising to 0.762 around the time of LT (p<0.001). Following LT, the median ACE score markedly dropped to 0.183 within a year and further dropped two years after LT, continuing to remain very low and comparable to the ACE scores for the control population. This trend remained consistent after controlling for medications commonly prescribed for complications of portal hypertension, such as nonselective beta-blockers, diuretics, and/or lactulose (FIG. 9).

### Example 9: ACE Scores in Asymptomatic Patients with Compensated Cirrhosis

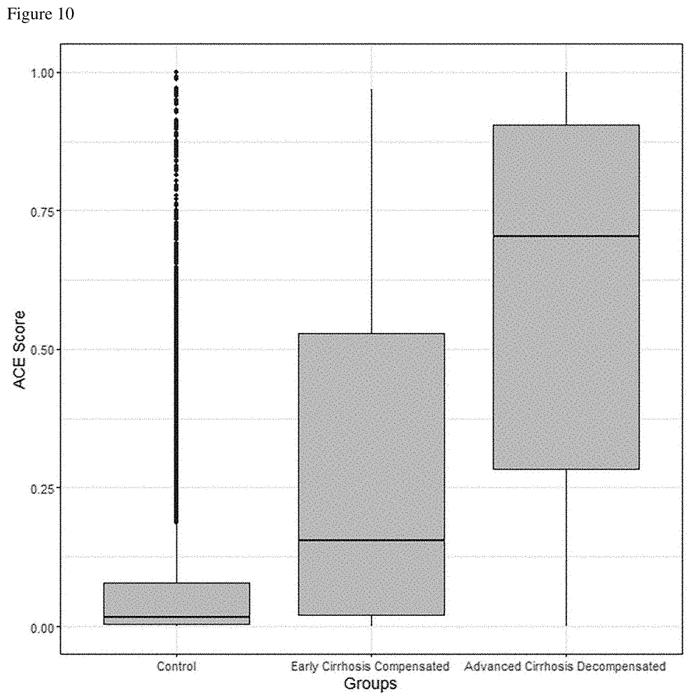

The CNN accurately classified most of the ECGs from the additional cohort of patients with compensated cirrhosis as having cirrhosis based on the established threshold of 0.17 from model development. The ACE scores for the compensated cirrhosis group (FIG. 10) were overall lower in comparison to the ACE scores of patients with decompensated disease, but also notably higher from non-cirrhosis controls. As shown in FIG. 10, Controls=Test set controls without cirrhosis. Early Cirrhosis Compensated=Patients with documented compensated cirrhosis without any decompensating events. ECGs were obtained within 6 months prior to the diagnosis of compensated cirrhosis. Advanced Cirrhosis Decompensated=Test set cirrhosis patients who underwent liver transplant.

### Example 10: ACE Score and Hepatic Decompensation

For patients in the retrospective cohort, the presence or absence of specific hepatic decompensation events (ascites, spontaneous bacterial peritonitis, variceal hemorrhage, hepatic encephalopathy, hepatorenal syndrome) at the time of each ECG was documented by manual chart review. Association between the ACE score and hepatic decompensation at the time of ECG was assessed using univariable and multivariable logistic regression adjusting for MELD-Na and covariates including demographics, comorbidities, and etiology of liver disease. In order to account for repeated ECG measures from the same patient, a random intercept was included to in the model for each unique patient. The ability of the ACE score to predict decompensation was also evaluated using a receiver-operating characteristics (ROC) curve and the resulting area under the ROC curve (AUC). A confidence interval for the AUC was calculated using a bootstrap resampling estimator. The optimal ACE cut-off for predicting decompensation was determined using Youden's index in order to provide a balance between sensitivity and specificity.

### Example 11: ACE Score and Liver-Related Outcomes

For both the prospective and retrospective cohorts, the ACE score was evaluated against liver-related outcomes including liver-related death and liver transplantation using competing risk Cox proportional hazards regression. ACE score was treated as a time dependent covariate due to the repeated ECG measures for each patient, allowing for a patient's ACE score to be updated in the Cox model with new ECGs. The primary competing outcomes of interest were liver transplant and liver-related death. All patients were censored at the time of last follow-up, death from any cause, and liver transplant. Cox regression models were univariable, multivariable adjusting for MELD-Na, and multivariable adjusting for patient demographics, comorbidities, liver etiology, and MELD-Na. In order to evaluate the associations between ACE score and liver-related outcomes in patients deemed to be low-risk by MELD-Na score, a sensitivity analysis was performed by restricting the cohort to only patients from the prospective cohort with a MELD-Na score of 15 or less. For Kaplan-Meier analysis, patients were divided into 4 quartiles based on their ACE score (0.00-0.25, 0.25-0.50, 0.50-0.75, and 0.75-1.00) and survival was compared using cumulative incidence curves for the competing outcomes.

### Example 12: Patient Characteristics of the Retrospective and Prospective Cohorts (ACE)

Table 5 provides an overview of patient characteristics in both of our study cohorts. There were 473 patients in the retrospective cohort who were initially diagnosed with compensated cirrhosis and followed over time, and 421 patients in the prospective cohort who were recruited at the time of evaluation for LT. The median age at the time of ECG was 64 in the retrospective cohort vs. 58 in the prospective cohorts. In both cohorts, there were more males than females and patients were predominantly White. Patients in the retrospective cohort had higher prevalences of hypertension, diabetes, and cardiopulmonary comorbidities while patients in the prospective cohort had a higher prevalence of chronic kidney diseases. The distribution of underlying liver disease etiologies is as shown.

TABLE 5
Baseline patient characteristics

| | Retrospective Cohort | | Prospective Cohort | |
|---|---|---|---|---|
| | (N = 473) | | (N = 421) | |
| Median Age at | 64 | (59, 72) | 58 | (50, 65) |
| ECG (Q1, Q3) | | | | |
| Sex | | | | |
| Male | 271 | (57.3%) | 236 | (56.1%) |
| Female | 202 | (42.7%) | 185 | (43.9%) |
| Race | | | | |
| American Indian/ | 5 | (1.1%) | 11 | (2.6%) |
| Alaskan Native | | | | |
| Asian | 9 | (1.9%) | 9 | (2.1%) |
| Black | 11 | (2.3%) | 8 | (1.9%) |
| White | 423 | (89.4%) | 353 | (83.8%) |
| Other/Did not disclose | 25 | (5.3%) | 40 | (9.5%) |
| Comorbidities | | | | |
| Coronary artery disease | 55 | (11.6%) | 31 | (7.4%) |
| Hypertension | 322 | (68.1%) | 213 | (50.6%) |
| Diabetes mellitus | 208 | (44.0%) | 150 | (35.6%) |
| Chronic kidney disease | 121 | (25.6%) | 188 | (44.7%) |
| Chronic lung disease | 197 | (41.6%) | 79 | (18.8%) |
| Etiology of Liver Disease | | | | |
| Viral | 138 | (29.2%) | 50 | (11.9%) |
| Alcohol-related | 110 | (23.3%) | 113 | (26.8%) |
| Non-alcoholic | 179 | (37.8%) | 122 | (29.0%) |
| steatohepatitis | | | | |
| Autoimmune | 30 | (6.3%) | 15 | (3.6%) |
| Biliary | 33 | (7.0%) | 62 | (14.7%) |
| Genetic/metabolic | 23 | (4.9%) | 10 | (2.4%) |
| Cryptogenic | 41 | (8.7%) | 47 | (11.2%) |

### Example 13: ACE Score and Hepatic Decompensation

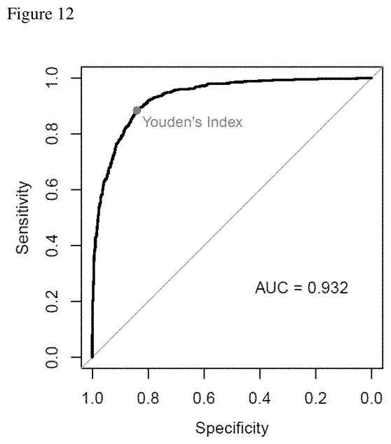

Among the 473 patients initially diagnosed with compensated cirrhosis (the retrospective cohort), 209 (44.9%) patients developed hepatic decompensation while 264 (55.1%) patients remained compensated during follow-up. FIG. 12 shows the ROC curve generated from using the ACE score as a binary classifier to determine the presence or absence of hepatic decompensation. The ACE score had an outstanding classification performance with an AUC of 0.932 (95% CI: 0.922-0.942). Using Youden's index, an optimal cut-off of 0.25 produced a sensitivity of 88.4% (95% CI: 86.2%-90.3%) and a specificity of 83.9% (95% CI: 81.6%-85.9%).

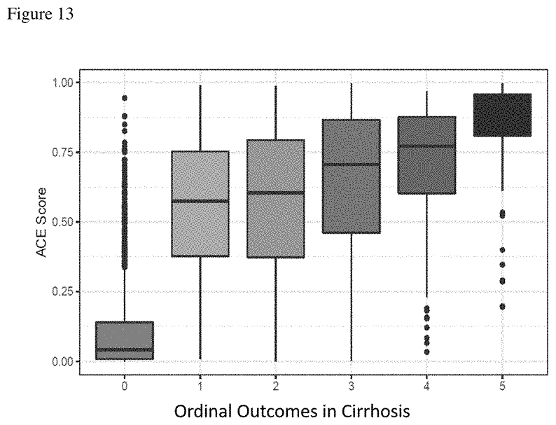

In addition to binary classification of compensated vs. decompensated state, the distribution ACE score was evaluated across levels of ordinal outcomes in cirrhosis as proposed by D'Amico et al (0=compensated, 1=development of varices, 2=variceal bleeding alone, 3=non-bleeding single decompensation, 4=more than one decompensating event, 5=end-stage complications leading to death). A progressive increase in the ACE score distribution was seen with the worsening degrees of hepatic decompensation (FIG. 13).

On univariate logistic regression, a 0.1-point increase in ACE score was associated with 4.3 times greater odds of decompensation (OR=4.28, 95% CI: 3.50-5.24, p<0.001). After adjusting the model for MELD-Na, a 0.1-point increase in ACE score was associated with 4.8 times increased odds of decompensation (OR=4.80, 95% CI: 3.60-6.40, p<0.001). Finally, in a full multivariable model adjusting for patient demographics, comorbidities, liver disease etiology, and MELD-Na, a 0.1-point increase in ACE score was still associated with almost twice greater odds of decompensation (OR=1.91, 95% CI: 1.43-2.55, p<0.001) (Table 6).

TABLE 6
Logistic regression for association between
ACE score and hepatic decompensation

| Model | Odds Ratio* | 95% CI | p-value |
|---|---|---|---|
| Unadjusted | 4.28 | 3.50-5.24 | <0.001 |
| Adjusted for | 4.80 | 3.60-6.40 | <0.001 |
| MELD-Na | | | |
| Adjusted for | | | |
| MELD-3.0 | | | |
| Fully adjusted** | 1.91 | 1.43-2.55 | <0.001 |

Abbreviations:
ACE, AI-Cirrhosis-ECG;
CI, confidence interval;
MELD-Na, model for end-stage liver disease-sodium
*For each 0.1-point increase in ACE score
**Adjusted for age, sex, race, medical comorbidities, etiology of liver disease, and MELD-Na.

### Example 14: ACE Score and Liver-Related Outcomes: Retrospective Cohort

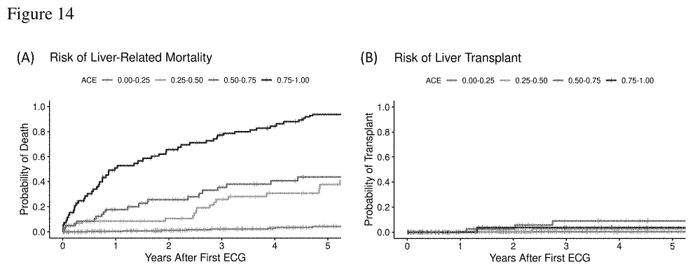

In the retrospective cohort of 473 patients initially diagnosed with compensated cirrhosis, a total of 122 (25.8%) patients experienced death due to liver-related causes and 34 (7.2%) patients received LT. FIGS. 14A-B shows the Kaplan-Meier curves for the competing time-to-event outcomes of liver-related death and LT. Significant increases in liver-related mortality were seen with increasing ACE quartiles over 5 years of follow up (FIG. 14A). The bottom quartile of patients (ACE 0.00-0.25) had a very low probability of death with over 90% still surviving 5 years after ECG, while patients in the top quartile (ACE 0.75-1.00) had extremely poor outcomes with around 50% mortality at 1 year, and over 90% mortality 5 years after ECG (p<0.001). On the other hand, no significant differences in the probability of LT were seen among the ACE quartiles (FIG. 14B).

On competing-risk Cox regression analysis (Table 7), the unadjusted univariable model found that a 0.1-point increase in ACE score was associated with 56% increased risk of liver-related death (HR=1.56, 95% CI: 1.46-1.66, p<0.001) and 34% increased risk of LT (HR=1.34, 95% CI: 1.12-1.61, p<0.001). After adjusting for MELD-Na, a 0.1-point increase in ACE score was still significantly associated with 35% increased risk of liver-related death (HR=1.35, 95% CI: 1.26-1.45, p<0.001), but no longer associated with probability of LT (HR=1.17, 95% CI: 0.96-1.42, p=0.129). In the full multivariable model, a 0.1-point increase in ACE score was associated with 42% increase in the risk of liver-related death (HR=1.42, 95% CI: 1.31-1.54, p<0.001) but not with probability of LT (HR=1.20, 95% CI: 0.97-1.48, p=0.088). In this cohort, ACE score was superior to MELD-Na for prediction of liver-related mortality (c-statistic=0.876 vs. 0.851, p<0.001). More importantly, combining MELD-Na with ACE led to significantly improved performance for liver-related mortality compared to either ACE or MELD-Na alone (c-statistic=0.911 vs. 0.876 vs. 0.851, p<0.001) (Table 8).

TABLE 7
Cox regression for association between
ACE score and liver-related outcomes

| | Liver-Related Death | | | Liver Transplant | | |
|---|---|---|---|---|---|---|
| | Hazards | | p- | Hazards | | p- |
| Model | Ratio* | 95% CI | value | Ratio* | 95% CI | value |

Retrospective cohort of patients initially
diagnosed with compensated cirrhosis

| | | | | | | |
|---|---|---|---|---|---|---|
| Unadjusted | 1.56 | 1.46- | <0.001 | 1.34 | 1.12- | <0.001 |
| | | 1.66 | | | 1.61 | |
| Adjusted for | 1.35 | 1.26- | <0.001 | 1.17 | 0.96- | 0.129 |
| MELD-Na | | 1.45 | | | 1.42 | |
| Adjusted for | | | | | | |
| MELD 3.0 | | | | | | |
| Fully | 1.42 | 1.31- | <0.001 | 1.20 | 0.97- | 0.088 |
| adjusted** | | 1.54 | | | 1.48 | |

Prospective cohort of patients
evaluated for liver transplantation

| | | | | | | |
|---|---|---|---|---|---|---|
| Unadjusted | 1.31 | 1.21- | <0.001 | 1.31 | 1.23- | <0.001 |
| | | 1.41 | | | 1.38 | |
| Adjusted for | 1.21 | 1.12- | <0.001 | 1.23 | 1.17- | <0.001 |
| MELD-Na | | 1.31 | | | 1.32 | |
| Adjusted for | | | | | | |
| MELD 3.0 | | | | | | |
| Fully | 1.19 | 1.10- | <0.001 | 1.25 | 1.18- | <0.001 |
| adjusted** | | 1.29 | | | 1.33 | |

Abbreviations:
ACE, AI-Cirrhosis-ECG;
CI, confidence interval;
MELD-Na, model for end-stage liver disease-sodium
*For each 0.1-point increase in ACE score
**Adjusted for age, sex, race, medical comorbidities, etiology of liver disease, and MELD-Na.

TABLE 8
Cox model concordances for prediction of liver-related outcomes

| | | | |
|---|---|---|---|
| | | Liver-Related | Liver |
| Model | Overall | Death | Transplant |

Retrospective cohort of patients initially
diagnosed with compensated cirrhosis

| | | | |
|---|---|---|---|
| ACE alone | 0.870 | 0.876 | 0.772 |
| MELD-Na alone | 0.851 | 0.851 | 0.856 |
| MELD 3.0 alone | | | |
| MELD-Na + ACE | 0.907 | 0.911 | 0.851 |
| MELD 3.0 + ACE | | | |

Prospective cohort of patients evaluated
for liver transplantation

| | | | |
|---|---|---|---|
| ACE alone | 0.693 | 0.680 | 0.698 |
| MELD-Na alone | 0.735 | 0.794 | 0.713 |
| MELD 3.0 alone | | | |
| MELD-Na + ACE | 0.779 | 0.818 | 0.764 |
| MELD 3.0 + ACE | | | |

Low MELD-Na (≤15) subgroup of
prospective cohort

| | | | |
|---|---|---|---|
| ACE alone | 0.749 | 0.786 | 0.739 |
| MELD-Na alone | 0.730 | 0.778 | 0.717 |
| MELD 3.0 alone | | | |
| MELD-Na + ACE | 0.809 | 0.871 | 0.793 |
| MELD 3.0 + ACE | | | |

### Example 15: ACE Score and Liver-Related Outcomes: Prospective Cohort

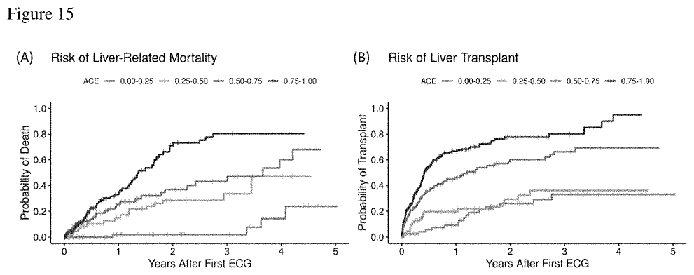

In the prospective cohort of 421 patients evaluated for LT, a total of 110 (26.1%) patients experienced death due to liver-related causes and 221 (52.5%) patients underwent successful LT. On Kaplan-Meier analysis, patients in higher ACE quartiles had significantly higher probabilities of death and liver transplantation compared to patients with lower ACE quartiles (FIGS. 15A-B).

On competing-risk Cox regression analysis (Table 7), a 0.1-point increase in ACE score was significantly associated with increased risk of liver-related death in the unadjusted univariable model (HR=1.31, 95% CI: 1.21-1.41, p<0.001), the model adjusted for MELD-Na (HR=1.21, 95% CI: 1.12-1.31, p<0.001), and the fully adjusted multivariable model (HR=1.19, 95% CI: 1.10-1.29, p<0.001). In this prospective cohort, a 0.1-point increase in ACE score was also associated with increased probability of liver transplantation in the unadjusted univariable model (HR=1.31, 95% CI: 1.23-1.38, p<0.001), the model adjusted for MELD-Na (HR=1.23, 95% CI: 1.17-1.31, p<0.001), and the fully adjusted multivariable model (HR=1.25, 95% CI: 1.18-1.33, p<0.001). In this cohort, the ACE score did not perform better than MELD-Na for prediction of liver-related mortality (c-statistic 0.680 vs. 0.794, p<0.001), but it still meaningfully improved the model performance to c-statistic of 0.818 when combined with MELD-Na (Table 8). The ACE score's ability to improve the predictive performance of MELD-Na was more pronounced in the sensitivity analysis of 110 patients who had MELD-Na of 15 or less. In this setting, the model combining ACE and MELD-Na significantly outperformed MELD-Na alone for prediction of liver-related mortality (c-statistic 0.871 vs. 0.778, p<0.001) (Table 8).

What is claimed:

1. A method comprising:
receiving voltage-time data of a subject, the voltage-time data comprising voltage data of a plurality of leads of an electrocardiograph;
generating a feature vector from the voltage-time data;
providing the feature vector to a pretrained learning system;
receiving from the pretrained learning system a status of liver disease in the subject.

2. The method of claim 1, wherein generating the feature vector comprises generating a spectrogram based on the voltage data of the plurality of leads.

3. The method of claim 1, wherein generating the feature vector comprises grouping the voltage data of the plurality of leads into a plurality of subsets.

4. The method of claim 1, further comprising:
receiving demographic information of the subject, wherein generating the feature vector comprises adding the demographic information to the feature vector.

5. The method of claim 1, further comprising:
receiving genomic information of the subject, wherein generating the feature vector comprises adding the genomic information to the feature vector.

6. The method of claim 1, wherein the learning system comprises a convolutional neural network.

7. The method of claim 6, wherein the convolutional neural network comprises at least one residual connection.

8. The method of claim 1, wherein the voltage-time data of a subject is received from an electrocardiograph.

9. The method of claim 1, wherein the voltage-time data of a subject is received from an electronic medical record.

10. The method of claim 1, further comprising:
providing the status to an electronic health record system for storage in a health record associated with the subject.

11. The method of claim 1, further comprising:
providing the status to a computing node for display to a user.

12. The method of claim 1, wherein the feature vector comprises a matrix having a plurality of rows and a plurality of columns, the plurality of rows corresponding to a temporal dimension and the plurality of columns corresponding to a spatial dimension.

13. The method of claim 12, wherein each of the plurality of rows correspond to one of the plurality of leads and each of the plurality of columns corresponds to a timestamp.

14. The method of claim 12, wherein the temporal dimension has a resolution of 500 Hz.

15. The method of claim 6, wherein the convolutional neural network comprises at least nine convolutional blocks and two fully connected blocks.

16. The method of claim 1, further comprising generating a liver disease outcome prediction for the patient.

17. The method of claim 1, wherein the pretrained learning system is trained by receiving a training set of voltage-time data from a plurality of cirrhosis patients.

18. The method of claim 17, wherein the training set of voltage-time data is from one of a retrospective cohort subset or a prospective cohort subset.

19. A system comprising:
an electrocardiograph comprising a plurality of leads;
a computing node comprising a computer readable storage medium having program instructions embodied therewith, the program instructions executable by a processor of the computing node to cause the processor to perform a method comprising:
receiving voltage-time data of a subject from the echocardiograph, the voltage-time data comprising voltage data of the plurality of leads;
generating a feature vector from the voltage-time data;
providing the feature vector to a pretrained learning system;
receiving from the pretrained learning system a status of liver disease in the subject.

20. The method of claim 19, wherein generating the feature vector comprises generating a spectrogram based on the voltage data of the plurality of leads.

21. The method of claim 19, wherein generating the feature vector comprises grouping the voltage data of the plurality of leads into a plurality of subsets.

22. The method of claim 19, further comprising:
receiving demographic information of the subject, wherein generating the feature vector comprises adding the demographic information to the feature vector.

23. The method of claim 19, further comprising:
receiving genomic information of the subject, wherein generating the feature vector comprises adding the genomic information to the feature vector.

24. The method of claim 19, wherein the learning system comprises a convolutional neural network.

25. The method of claim 24, wherein the convolutional neural network comprises at least one residual connection.

26. The method of claim 19, wherein the voltage-time data of a subject is received from an electrocardiograph.

27. The method of claim 19, wherein the voltage-time data of a subject is received from an electronic medical record.

28. The method of claim 19, further comprising:
providing the status to an electronic health record system for storage in a health record associated with the subject.

29. The method of claim 19, further comprising:
providing the status to a computing node for display to a user.

30. The method of claim 19, wherein the feature vector comprises a matrix having a plurality of rows and a plurality of columns, the plurality of rows corresponding to a temporal dimension and the plurality of columns corresponding to a spatial dimension.

31. The method of claim 30, wherein each of the plurality of rows correspond to one of the plurality of leads and each of the plurality of columns corresponds to a timestamp.

32. The method of claim 30, wherein the temporal dimension has a resolution of 500 Hz.

33. The method of claim 24, wherein the convolutional neural network comprises at least nine convolutional blocks and two fully connected blocks.

34. The method of claim 19, further comprising generating a liver disease outcome prediction for the patient.

35. The method of claim 19, wherein the pretrained learning system is trained by receiving a training set of voltage-time data from a plurality of cirrhosis patients.

36. The method of claim 35, wherein the training set of voltage-time data is from one of a retrospective cohort subset or a prospective cohort subset.

37. A computer program product for detection of liver disease, the computer program product comprising:
a computer readable storage medium having program instructions embodied therewith, the program instructions executable by a processor to cause the processor to perform a method comprising:
receiving voltage-time data of a subject from the echocardiograph, the voltage-time data comprising voltage data of the plurality of leads;
generating a feature vector from the voltage-time data;
providing the feature vector to a pretrained learning system;
receiving from the pretrained learning system a status of liver disease in the subject.

38. The method of claim 37, wherein generating the feature vector comprises generating a spectrogram based on the voltage data of the plurality of leads.

39. The method of claim 37, wherein generating the feature vector comprises grouping the voltage data of the plurality of leads into a plurality of subsets.

40. The method of claim 37, further comprising:
receiving demographic information of the subject, wherein generating the feature vector comprises adding the demographic information to the feature vector.

41. The method of claim 37, further comprising:
receiving genomic information of the subject, wherein generating the feature vector comprises adding the genomic information to the feature vector.

42. The method of claim 37, wherein the learning system comprises a convolutional neural network.

43. The method of claim 42, wherein the convolutional neural network comprises at least one residual connection.

44. The method of claim 37, wherein the voltage-time data of a subject is received from an electrocardiograph.

45. The method of claim 37, wherein the voltage-time data of a subject is received from an electronic medical record.

46. The method of claim 37, further comprising:
providing the status to an electronic health record system for storage in a health record associated with the subject.

47. The method of claim 37, further comprising:
providing the status to a computing node for display to a user.

48. The method of claim 37, wherein the feature vector comprises a matrix having a plurality of rows and a plurality of columns, the plurality of rows corresponding to a temporal dimension and the plurality of columns corresponding to a spatial dimension.

49. The method of claim 48, wherein each of the plurality of rows correspond to one of the plurality of leads and each of the plurality of columns corresponds to a timestamp.

50. The method of claim 48, wherein the temporal dimension has a resolution of 500 Hz.

51. The method of claim 42, wherein the convolutional neural network comprises at least nine convolutional blocks and two fully connected blocks.

52. The method of claim 37, further comprising generating a liver disease outcome prediction for the patient.

53. The method of claim 37, wherein the pretrained learning system is trained by receiving a training set of voltage-time data from a plurality of cirrhosis patients.

54. The method of claim 53, wherein the training set of voltage-time data is from one of a retrospective cohort subset or a prospective cohort subset.
